(19) United States
(12) Patent Application Publication
(10) Pub. No.: US 2011/0202094 A1
(43) Pub. Date: Aug. 18, 2011

(54) TRANS-POLYAXIAL SCREW

(76) Inventors: Mario L. PEREIRA, Port Charlotte, FL (US); James Scott HAY, Parkland, FL (US); Ryan SINGH, Loxahatchee, FL (US)

(21) Appl. No.: 12/944,494

(22) Filed: Nov. 11, 2010

Related U.S. Application Data
(60) Provisional application No. 61/260,206, filed on Nov. 11, 2009.

(51) Int. Cl. A61B 17/86

(52) U.S. Cl. 606/305

(57) ABSTRACT

An orthopedic screw, including a threaded shaft defining a longitudinal axis; a head defining a region able to receive at least a portion of a prosthesis, the head being coupled to the shaft such that the head is movably offset from the longitudinal axis in a direction substantially perpendicular to the longitudinal axis; and a spacing element positionable within the head, the spacing element defining a first alignment marker positionable adjacent a second alignment marker defined by the head.

### CROSS-REFERENCE TO RELATED APPLICATION

This application is related to and claims priority to U.S. Provisional Patent Application Ser. No. 61/260,206, filed Nov. 11, 2009, entitled “TRANS-POLYAXIAL SCREW,” the entirety of which is incorporated herein by reference.

### STATEMENT REGARDING FEDERALLY SPONSORED RESEARCH OR DEVELOPMENT

n/a

### FIELD OF THE INVENTION

The present invention relates to orthopedic anchors and fixation devices, and in particular, to a screw having a laterally or trans-axially adjustable segment.

### BACKGROUND OF THE INVENTION

Many types of spinal irregularities can cause pain, limit range of motion, or injure the nervous system within the spinal column. These irregularities can result from trauma, tumor, disc degeneration, and disease. Often, these irregularities are treated by immobilizing a portion of the spine. This treatment typically involves affixing a plurality of screws and/or hooks to one or more vertebrae and connecting the screws or hooks to elongate rods or other prostheses that generally extend in the direction of the axis of the spine to stabilize or otherwise limit a range of motion of the effected spinal segment.

In particular, treatment for these spinal irregularities often involves using a system of pedicle screws and rods to attain stability between adjacent spinal segments. Instability in the spine can create stress and strain on neurological structures, such as the spinal cord and nerve roots. In order to correct this, implants of certain stiffness can be implanted to restore the correct alignment and portion of the vertebral bodies. In many cases, an anchoring member such as a pedicle screw along with a vertical solid member can help restore spinal elements to a pain free situation, or at least may help reduce pain or prevent further injury to the spine.

It is well known that difficulties can arise during a surgical procedure when attempting to connect a solid, linear member (such as a rod) to a bone screw. As a result, it may be desirable to use a screw having an implant/rod-receiving head that is pivotable about the threaded screw shaft. The angular range of motion of so-called polyaxial screws reduces the difficulty in coupling a rod or implant to the screw. Yet difficulties remain in aligning a coupling rod or implant to such screws. While a typical polyaxial screw may provide some degree of angular manipulation of the screw head to engage the rod, there may be difficulty adjusting or engaging the screw head at the desired depth or transverse (e.g., medial-to-lateral and vice versa) positioning of the rod. Accordingly, it is desirable to provide a screw or anchoring system that provides both angular and/or rotational degrees of freedom as well as an adjustable or selectable transverse positioning of the rod within a portion of the head of the screw.

### SUMMARY OF THE INVENTION

The present invention advantageously provides an anchoring system that provides both angular and/or rotational degrees of freedom as well as an adjustable or selectable transverse positioning of one segment of the anchor with another segment.

An implantable anchoring system is provided, including an elongate member defining a threaded segment and a longitudinal axis; and a head defining an opening for receiving at least a portion of a prosthesis, the head being slidably positionable with respect to the elongate member in a direction substantially transverse to the longitudinal axis. The head portion may be movably coupled to the elongate member to define a ball-and-socket joint; the elongate member may define a passage therethrough substantially parallel to the longitudinal axis; and/or the elongate member may define a tool engagement feature. The system may include a spacing element positionable within the head between the elongate member and the opening in the head for receiving the prosthesis, where the spacing element may define an opening therethrough substantially perpendicular to the longitudinal axis, the opening positionable adjacent to a second opening in the head. The system may include a pin insertable into the second opening in the head and the opening in the spacing element, where the spacing element may be movable about the pin in a direction substantially parallel to the longitudinal axis. The system may include a collar affixed to the elongate member and positionable within the head, where the collar defines a first tool engagement feature alignable with a second tool engagement feature defined by the head. The first tool engagement feature may include at least one of a cylindrical passage in the collar or a semi-cylindrical indentation on an exterior circumference of the collar; and the second tool engagement feature may include a cylindrical passage through a portion of the head. The system may include a tool engageable with the first and second tool engagement features and a spacing element positionable within the head, the spacing element defining a third tool engagement feature alignable with the second tool engagement feature, where the third tool engagement feature includes a cylindrical passage through the spacing element.

An orthopedic screw is provided, including a threaded shaft defining a longitudinal axis; and a head defining a region able to receive at least a portion of a prosthesis, the head being coupled to the shaft such that the head is movably offset from the longitudinal axis in a direction substantially perpendicular to the longitudinal axis. The screw may include a spacing element positionable within the head, the spacing element defining a first alignment marker positionable adjacent a second alignment marker defined by the head.

A method of implanting a prosthesis is provided, including positioning an anchor in proximity to a surgical site, the anchor including a shaft defining a longitudinal axis and a head movably coupled to the shaft; rotatably securing a threaded portion of a shaft into a tissue site; slidably positioning the head with respect to the shaft such that the head is offset from the longitudinal axis in a direction substantially perpendicular to the longitudinal axis; and coupling the prosthesis to the head. The method may include positioning a spacing element between the shaft and the prosthesis; aligning a first opening defined by the spacing element with a second opening defined by the head; and/or rotatably engaging a set screw to the head to secure the prosthesis to the anchor.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is an illustration of an implantable anchoring system constructed in accordance with the principles of the present invention;

FIG. 2 is a cross-sectional view of the implantable anchoring system shown in FIG. 1;

FIG. 2A is another cross-sectional view of the implantable anchoring system shown in FIG. 1;

FIG. 3 is an additional cross-sectional view of the implantable anchoring system shown in FIG. 1;

FIG. 4 is an illustration of another implantable anchoring system constructed in accordance with the principles of the present invention;

FIG. 5 is a cross-sectional view of the implantable anchoring system shown in FIG. 4; and

FIG. 6 is an additional cross-sectional view of the implantable anchoring system shown in FIG. 4.

### DETAILED DESCRIPTION OF THE INVENTION

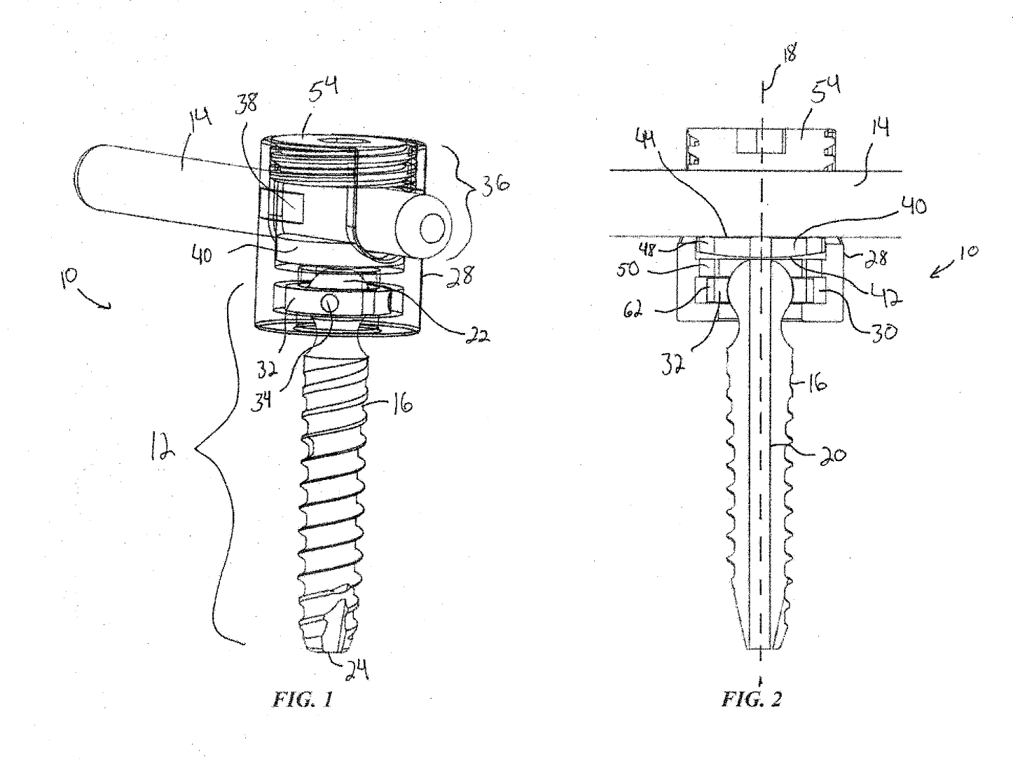

The present invention provides an anchoring system that provides both angular and/or rotational degrees of freedom as well as an adjustable or selectable transverse positioning of one segment of the anchor with another segment. Referring now to the drawing figures in which like reference designations refer to like elements, an anchoring system constructed in accordance with the principles of the present invention is shown in FIGS. 1-6 and generally designated as “10.” Of note, the system components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein. Moreover, while certain embodiments or figures described herein may illustrate features not expressly indicated on other figures or embodiments, it is understood that the features and components of the system and components disclosed herein are not necessarily exclusive of each other and may be included in a variety of different combinations or configurations without departing from the scope and spirit of the invention.

The anchoring system 10 may generally include a fastener or anchor 12, such as an orthopedic screw that is insertable or implantable into a tissue region or surgical site to secure at least a portion of an implantable prosthesis 14 to that tissue region or surgical site. The implantable prosthesis 14 may include, for example, an artificial device or component that replaces, augments, or supplements a missing, defective, or debilitated part of the body. Such prostheses may include, for example, a spinal stabilization rod, a motion-limiting or supporting device, or the like.

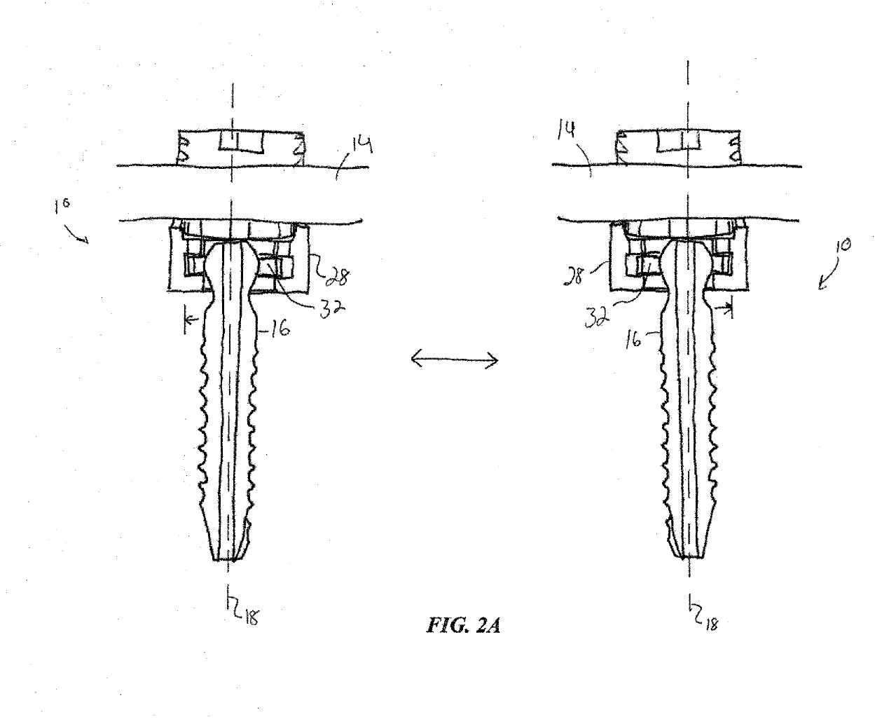

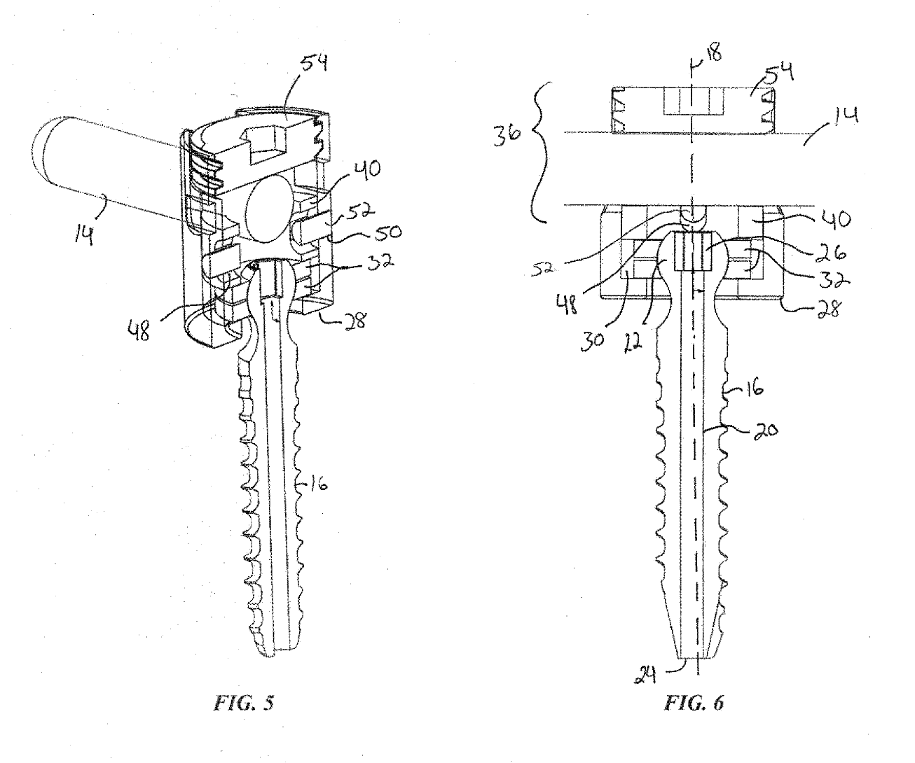

The anchor 12 may include an elongate body or shaft 16 defining a threaded segment along at least a portion of the length of the shaft 16. As shown in FIGS. 2-3 and 5-6, the elongate body may further define a longitudinal axis 18 and a passage 20 extending through the shaft 16 substantially parallel to the axis 18. The passage 20 may allow for the introduction of rehabilitative materials that aid in securing the anchor 12 to the tissue (such as an adhesive or cement) and/or facilitate recovery of the surgical site (such as biological or pharmaceutical healing/growth-promoting agents). The shaft 16 may include a first end 22 having a rounded or spherical shape that is opposite a second end 24 of the shaft 16 designated for insertion into the tissue. The first end 22 may define a tool engagement feature 26 that eases or facilitates manipulation of the shaft 16 with a complementary external tool, such as a screwdriver or the like. For example, the tool engagement feature 26 may include a hex socket (as shown in FIG. 5) engageable with one or more socket wrenches or drivers.

The anchor 12 may also include a head 28 attached to the first end of the shaft 16. The anchor 12 may be movable about the shaft 16 along a plurality of axes. For example, the head 28 may form a ball-and-socket joint with the rounded or contoured first end 22 of the shaft 16 that allows the head 28 to rotate and pivot about the first end 22 of the shaft 16 in a plurality of directions. The head 28 may also be slidably or otherwise movably positioned about the first end 22 of the shaft 16 in a direction substantially transverse or perpendicular to the longitudinal axis 18. For example, the cross-sectional view of the anchoring system 10 in FIG. 2 illustrates the shaft 16 substantially centered with respect to a width of the head 28. Referring now to FIG. 2A, the shaft 16 and head 28 are illustrated as movably or slidably offset with respect to one another along a direction transverse to the axis 18. The offset, transverse range of motion for the translation between the shaft 16 and the head 28 provides an additional degree of manipulation to align one or more anchors with one or more prostheses during a surgical procedure, which eases the overall procedure for an operating physician and may further reduce an overall time of the procedure.

The transverse movement or translation between the head 28 and the shaft 16 may be accomplished by providing a clearance or space for movement between the shaft 16 and the head 28. For example, the head 28 may define a cavity or compartment 30 for receiving at least a portion of the first end of the shaft 16. One or more washers or collars 32 may be coupled to the first end of the shaft 16 to facilitate placement and/or engagement of the shaft 16 to the head 28. The collar(s) 32 may be either immovably fixed to the first end of the shaft 16 by one or more locking pins 34 (as shown in FIG. 1), or may be coupled to the first end of the shaft 16 to allow multiple degrees of freedom and/or motion between the collar(s) 32 and the shaft 16, as shown in FIGS. 4-6. The collar(s) 32 may generally define an outer dimension or circumference that is smaller than an interior circumference or dimension of the compartment 30, thus allowing the collar(s) 32 and the shaft 16 to slide or move within the compartment in directions transverse to the longitudinal axis 18. For example, if the shaft 16 is implanted into a portion of the spinal column, the longitudinal axis 18 may be generally or substantially parallel to a posterior-to-anterior axis of the patient. The head 28 would thus be movable about the shaft 16 (and the axis) in both caudal-to-cranial directions as well as medial-to-lateral directions resulting in an offset or asymmetrical alignment about the axis 18 in any of those directions.

The head 28 may also define a region 36 for receiving at least a portion of the prosthesis 14. For example, the head 28 may define a cavity defined between two substantially “U”-shaped apertures in an outer wall of the head 28. The prosthesis 14 may be at least partially positioned within the cavity such that it extends through that segment of the head 28 and through the U-shaped openings. The head 28 may also define one or more indentations 38 on its exterior surface that facilitate engagement with an external tool to insert, position, and/or align the anchor 12 or a portion thereof.

The anchoring system 10 may also include one or more spacing elements 40 movably positionable within a portion of the head 28 between the prosthesis 14 and the shaft 16. The spacing element(s) 40 may generally aid in the positioning or coupling of the prosthesis 14 to the head 28 of the anchor 12. The spacing element 40 may include or define a prosthesis-receiving or mating feature that is complementary to a shape, feature, or dimension of the prosthesis 14. For example, the spacing element 40 may generally define a cylindrical or circular body having a first side 42 that faces or contacts the shaft 16, and a second side 44 that faces or otherwise contacts the prosthesis 14. The second side 44 that faces or receives a portion of the prosthesis 14 may define a rounded or contoured depression 46. The depression 46 may have a radius of curvature or other matable feature substantially similar to a radius of curvature or feature defined by a portion of the prosthesis 14 placed within or traversing the head 28 of the anchor 12.

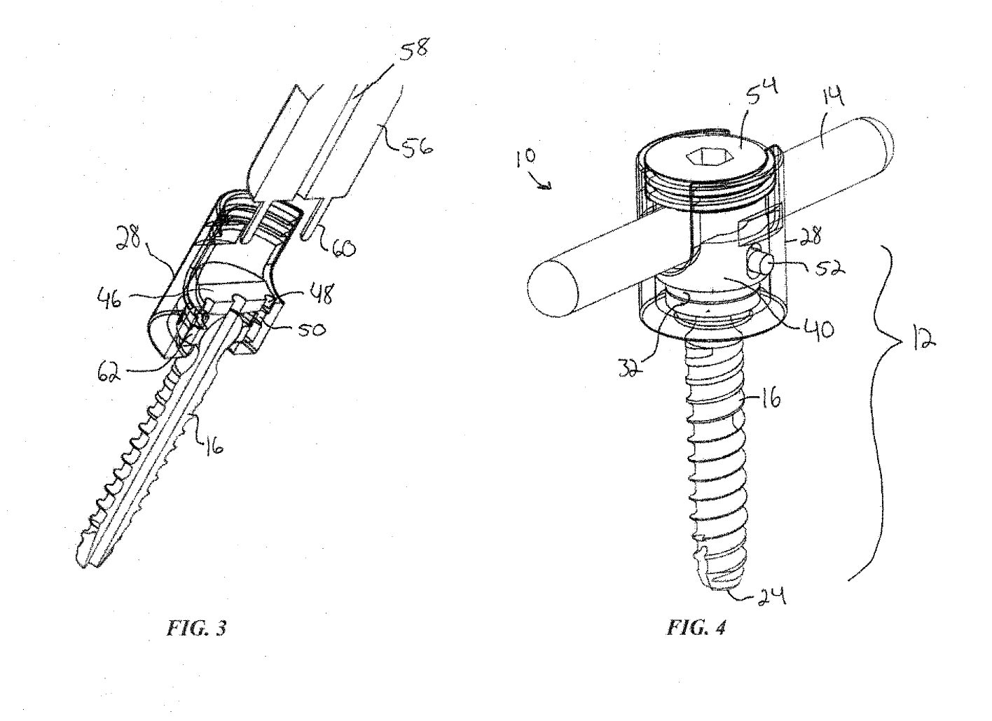

The spacing element 40 may also include an alignment marking or feature that ensures the desired orientation of the spacing element within the head 28 of the anchor 12. The alignment marking or feature can allow precise positioning of the prosthesis 14-receiving or mating feature (i.e., the depression for example) in alignment with the prosthesis 14-receiving region of the head 28. For example, as shown in FIGS. 2-3, the alignment feature or marker may include one or more passages 48 defined by the spacing element 40 extending therethrough in a direction substantially parallel to the axis 18. Alternatively, the passages 48 may be oriented in a direction substantially perpendicular or transverse to the axis 18, as shown in FIGS. 4-5. The passages 48 may be positioned or matched to associated alignment features or markers on the head 28 of the anchor 12. For example, the head 28 of the anchor 12 may define one or more passages 50 therethrough. The passages 50 may be oriented substantially parallel to the longitudinal axis (as shown in FIG. 3) or perpendicular to the axis 18 (as shown in FIG. 5) to align with or be positioned adjacent to the respectively oriented passage(s) 48 of the spacing element 40. The alignment between the spacing element 40 and the head 28 may be achieved through the use of a tool that can be directed through the passages 48, 50 to ensure their alignment, as discussed below. Additionally, as shown in FIGS. 4-5, one or more pins 52 may be inserted through the passage 50 of the head 28 and through at least a portion of the passage 48 of the spacing element 40 to ensure the alignment between the two components and to prevent movement between the anchor 12 and the spacing element 40 once secured.

The spacing element 40 can provide a desired offset or spacing between the prosthesis 14 within the head 28 and/or in relation to the shaft 16. For example, the system 10 may include a plurality of selectable interchangeable spacing elements 40, with each having a varied predefined thickness to provide the desired offset between the prosthesis 14 and the shaft 16 (and thus the tissue) for a particular surgical procedure or patient. The spacing element 40 may also be selectively positionable in a plurality of securable positions within the head 28 to provide the desired spacing, offset, or height with respect to the shaft 16. For example, referring to FIGS. 4-5, the passage 48 may have an increased diameter or dimension as compared to the intersecting pin 52, allowing an increased range of positions or heights while still allowing engagement with the pin 52. The spacing element 40 can thus be positioned at a desired height or position within the head 28, and the pin 52 can be inserted or coupled to the spacing element to prevent any further movement between the components. Alternatively, the spacing element 40 may be slidable along the pin 52. The slidable coupling may be friction or compression fit requiring a threshold of effort to move the spacing element 40, thus providing sufficient stability for implanted use once a selected position is achieved.

The anchoring system 10 may further include a locking component 54, such as a set screw, engageable with the head 28 to secure the prosthesis 14 to the anchor 12. The head 28 may define a threaded segment complementary to a threaded portion of the locking component 54. Securing the locking component 54 to the head 28 may result in securing or compressing the head 28, spacing element 40, and the shaft 16 together to restrict any further movement once the locking component 54 is installed.

The anchoring system 10 may further include an instrument or tool 56 that aids in the insertion and/or positioning of one or more components of the system 10. The instrument 56 may define a channel 58 extending through at least a portion of its length that is positionable adjacent to or otherwise alignable with the passage 20 of the shaft 16. The instrument 56 thus allows the introduction of materials or introduction of other auxiliary instruments through the instrument 56 and into the passage 20 of the shaft 16.

The instrument 56 may include a feature or characteristic that is complementary and/or releasably engageable with one or more tool-engagement features of the head 28, spacing element 40, collar 32, and/or shaft 16. For example, now referring to FIG. 3, the instrument 56 may include one or more protrusions 60 engageable with the shaft 16, collars 32, spacing element 40, and/or head 28 of the anchor 12. The protrusions 60 may be releasably engageable with the passages 48 of the spacing element 40 and/or the passages 50 of the head 28. In addition, the collar 32 may define one or more passages 62 therethrough that can engage the instrument 56, as shown in FIGS. 2-3. The passages 62 of the collar may be rotatably positionable within the head 28 to align with the passages 50 in the head 28. The instrument 56 can thus be used to engage both the head 28 and the collar 32 to drive or rotate the shaft 16 into a tissue region. Once the shaft 16 is desirably positioned, the instrument 56 can be removed such that the head 28 is freely rotatable, pivotable, or transversely adjustable about the shaft 16. The spacing element 40 can then be introduced into the head 28. The instrument 56 can then be used to align the spacing element 40 with the head 28 by aligning the passages 48 of the spacing element 40 with the passages 50 of the head 28 using the protrusions 60 of the instrument 56.

Of note, while the matable or complementary engagement between the instrument and the other components of the anchor 12ing system 10 are described and illustrated as protrusions coupling to respective passages in the head 28, spacing element, and/or collar, it is contemplated that other complementary, releasably engageable modalities may be used without departing from the described operational relationship between the components.

By providing an anchoring system that provides both angular and/or rotational degrees of freedom as well as an adjustable or selectable transverse positioning of the rod within a portion of the head of the screw, variations in implantable prosthesis geometry or positioning as well as anatomical differences between patients can be readily accommodated with a single system.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. An implantable anchoring system, comprising:
an elongate member defining a threaded segment and a longitudinal axis; and
a head defining an opening for receiving at least a portion of a prosthesis, the head being slidably positionable with respect to the elongate member in a direction substantially transverse to the longitudinal axis.

2. The anchoring system of claim 1, wherein the head portion is movably coupled to the elongate member to define a ball-and-socket joint.

3. The anchoring system of claim 1, wherein the elongate member defines a passage therethrough substantially parallel to the longitudinal axis.

4. The anchoring system of claim 1, wherein the elongate member defines a tool engagement feature.

5. The anchoring system of claim 1, further comprising a spacing element positionable within the head between the elongate member and the opening in the head for receiving the prosthesis.

6. The anchoring system of claim 5, wherein the spacing element defines an opening therethrough substantially perpendicular to the longitudinal axis, the opening positionable adjacent to a second opening in the head.

7. The anchoring system of claim 6, further comprising a pin insertable into the second opening in the head and the opening in the spacing element.

8. The anchoring system of claim 7, wherein the spacing element is movable about the pin in a direction substantially parallel to the longitudinal axis.

9. The anchoring system of claim 1, further comprising a collar affixed to the elongate member and positionable within the head.

10. The anchoring system of claim 9, wherein the collar defines a first tool engagement feature alignable with a second tool engagement feature defined by the head.

11. The anchoring system of claim 10, wherein the first tool engagement feature includes at least one of a cylindrical passage in the collar or a semi-cylindrical indentation on an exterior circumference of the collar; and the second tool engagement feature includes a cylindrical passage through a portion of the head.

12. The anchoring system of claim 10, further comprising a tool engageable with the first and second tool engagement features.

13. The anchoring system of claim 10, further comprising a spacing element positionable within the head, the spacing element defining a third tool engagement feature alignable with the second tool engagement feature.

14. The anchoring system of claim 13, wherein the third tool engagement feature includes a cylindrical passage through the spacing element.

15. An orthopedic screw, comprising:
a threaded shaft defining a longitudinal axis; and
a head defining a region able to receive at least a portion of a prosthesis, the head being coupled to the shaft such that the head is movably offset from the longitudinal axis in a direction substantially perpendicular to the longitudinal axis.

16. The orthopedic screw of claim 15, further comprising a spacing element positionable within the head, the spacing element defining a first alignment marker positionable adjacent a second alignment marker defined by the head.

17. A method of implanting a prosthesis, comprising:
positioning an anchor in proximity to a surgical site, the anchor including a shaft defining a longitudinal axis and a head movably coupled to the shaft;
rotatably securing a threaded portion of a shaft into a tissue site;
slidably positioning the head with respect to the shaft such that the head is offset from the longitudinal axis in a direction substantially perpendicular to the longitudinal axis; and
coupling an implantable prosthesis to the head.

18. The method of claim 17, further comprising positioning a spacing element between the shaft and the prosthesis.

19. The method of claim 18, further comprising aligning a first opening defined by the spacing element with a second opening defined by the head.

20. The method of claim 16, further comprising rotatably engaging a set screw to the head to secure the prosthesis to the anchor.
